(19) United States
(12) Patent Application Publication
(10) Pub. No.: US 2024/0132691 A1
(43) Pub. Date: Apr. 25, 2024

(54) METHODS FOR PRODUCING SYNTHETIC OLIGONUCLEOTIDES AND REMOVAL AGENTS FOR REMOVING IMPURITIES FROM ORGANIC WASH SOLVENTS USED IN OLIGONUCLEOTIDE SYNTHESIS

(71) Applicant: Honeywell International Inc., Charlotte, NC (US)

(72) Inventors: Jüergen Rudolph, Peine (DE); James Guiheen, Madison, NJ (US); Sandra Lorenz, Grand Haven, MI (US); Stefan Seekamp, Wunstorf (DE); Christian Werner, Hannover (DE); Jared OConnell, New York, NY (US)

(21) Appl. No.: 18/225,010

(22) Filed: Jul. 20, 2023

Related U.S. Application Data
(60) Provisional application No. 63/392,441, filed on Jul. 26, 2022.

(51) Int. Cl. C08J 11/02; C07H 21/00

(52) U.S. Cl. CPC C08J 11/02; C07H 21/00

(57) ABSTRACT

The present invention provides methods for utilizing blended compositions of acetonitrile and toluene as organic wash solvents in the production of a synthetic oligonucleotide, the blended compositions producing higher synthetic oligonucleotide yields than a pure acetonitrile wash solvent. The method also provides a process for removing one or more impurities from the acetonitrile and toluene containing wash solvent received as a waste stream from the oligonucleotide synthesis process. The process includes adding at least one of an iodine reactive compound, a sulfur reactive compound and/or an acidic reactive compound to the waste stream, and fractionating the waste stream. The fractionation produces an overhead fraction and a bottom fraction where the overhead fraction includes the acetonitrile and the toluene, and the bottom fraction includes the one or more impurities.

### CROSS-REFERENCE TO RELATED APPLICATION

This application claims the benefit of U.S. Provisional Application No. 63/392,441, filed Jul. 26, 2022, which is herein incorporated by reference in its entirety.

### FIELD

The technical field generally relates to methods and systems for purifying a waste stream including acetonitrile and toluene, and more particularly, the addition of one or more reactive compounds to the waste stream, which enables the recovery and subsequent recycling of a substantially purified mixture of acetonitrile and toluene. In some cases, the recycled and purified mixture of acetonitrile and toluene yields higher purity oligonucleotides, as compared to pure acetonitrile, when utilized as a wash solvent in oligonucleotide synthesis processes.

### BACKGROUND

Numerous chemical processes utilize acetonitrile as a solvent or wash, resulting in generation of low grade acetonitrile waste streams. When these processes are conducted on a manufacturing scale, the volume of low grade acetonitrile waste stream produced can be substantial. For example, oligonucleotide (DNA and RNA) synthesis is generally conducted via a four step cycle (deblocking, activation/coupling, capping, and oxidation), which is repeated for each nucleotide added until the desired sequence is obtained. Between each step, oligonucleotides bound to a support are typically washed with high purity (i.e., 100%) acetonitrile to reduce residual reagents from the prior step. This leads to generation of a large volume of acetonitrile waste stream, wherein approximately 2,000 metric tons of acetonitrile are used when manufacturing approximately 1 metric ton of an oligonucleotide based active pharmaceutical ingredients (APIs).

Synthetic oligonucleotide sequences are showing promise for therapeutic, diagnostic, and drug target validation applications in the bio pharmaceutical industry, and the number of oligonucleotide-based drugs currently in pre-clinical or clinical trials is ever increasing. However, oligonucleotide based API manufacturers are faced with the expense and difficulty of managing both the procurement of large quantities of pure acetonitrile, as well as the disposition of large volume of acetonitrile-based waste stream produced. As such, there is an acute need in the industry for methods and systems to reclaim and purify acetonitrile waste streams to generate acetonitrile-based wash solvent suitable for reuse. Furthermore, other desirable features and characteristics will become apparent from the subsequent detailed description and the appended claims, taken in conjunction.

### SUMMARY

The present disclosure provides processes and methods for removal of one or more impurities, including iodine-containing compounds, sulfur containing compounds, and/or basic nitrogen containing compounds, from a waste stream generated by an oligonucleotide process. In this case, the waste stream contains acetonitrile and toluene at a compositional ratio that is rich in Acetonitrile as well as one or more impurities. One or more reductants/scavengers, including an iodine reactive compound, a sulfur reactive compound and/or an acidic reactive compound, are added to the waste stream, and during fractionation, the overhead fraction includes the acetonitrile and toluene at an azeotropic composition, and the bottom fraction contains the one or more impurities. The overhead fraction may be further processed to remove any remaining impurities, and the purified acetonitrile and toluene mixture can be recycled into the oligonucleotide process as a wash solvent.

In one embodiment, the present disclosure provides a method for processing a waste stream including receiving the waste stream including acetonitrile, toluene, and one or more iodine containing compounds, adding an iodine reactive compound to the waste stream, and fractionating the waste stream to produce an overhead fraction and a bottom fraction. In this case, the overhead fraction includes the acetonitrile and the toluene of the waste stream, and the bottom fraction comprising the one or more iodine containing compounds of the waste stream.

In another embodiment, the present disclosure provides a method for processing a waste stream including receiving the waste stream including acetonitrile, toluene, and one or more sulfur containing compounds, adding a sulfur reactive compound to the waste stream, and fractionating the waste stream to produce an overhead fraction and a bottom fraction. In this case, the overhead fraction includes the acetonitrile and toluene of the waste stream, and the bottom fraction includes the one or more sulfur containing compounds of the waste stream.

In yet another embodiment, the present disclosure provides a method for processing a waste stream including receiving the waste stream including acetonitrile, toluene, and basic nitrogen containing compounds, adding an acidic reactive compound to the waste stream and fractionating the waste stream to produce an overhead fraction and a bottom fraction. In this case, the overhead fraction includes the acetonitrile and toluene of the waste stream and the bottom fraction includes the basic nitrogen containing compounds of the waste stream.

In a further embodiment, the present disclosure provides a method for producing a synthetic oligonucleotide including washing a reaction vessel containing one or more components of an oligonucleotide synthesis process with a blended wash solution of at least acetonitrile and toluene, where the acetonitrile is at least 70% of the blended wash solution, and recovering the synthetic oligonucleotide. In this case, the synthetic oligonucleotide is recovered at a minimum oligonucleotide purity.

### BRIEF DESCRIPTION OF THE DRAWINGS

FIG. 1 is a schematic illustration of a system and method for purifying a waste stream in accordance with an exemplary embodiment.

### DETAILED DESCRIPTION

The following detailed description is merely exemplary in nature and is not intended to limit the various embodiments or the application and uses thereof. Furthermore, there is no intention to be bound by any theory presented in the preceding background or the following detailed description.

Methods, systems, and processes for purifying an oligonucleotide waste stream are provided herein. The process, methods, and systems of the present invention offer a technically simplified, and economically advantageous way, of pre-treating and recycling a waste stream received from oligonucleotide syntheses. As described previously, oligonucleotide synthesis processes (e.g., solid-phase synthesis) utilize a substantial amount of acetonitrile-based solvent as a wash solvent, and during such processes, the acetonitrile-based solvent becomes contaminated by many impurities.

Oligonucleotide syntheses processes typically require a pure acetonitrile wash solvent, and as such, the acetonitrile containing waste stream cannot be reused. Therefore, there is a need to purify the acetonitrile containing waste stream to a level acceptable for recycling back into the synthesis process. It has been found that such synthesis processes contaminate the acetonitrile with multiple compounds, including toluene (e.g., as contaminated via a deblocking agent), iodine (e.g., as contaminated by an oxidation reagent), sulfur containing compounds such as thiols (e.g., as contaminated by the sulfurization and activation reagents) and basic nitrogen containing compounds such as alkylamines (e.g., as contaminated by the oxidation, capping and activation reagents). In this case, it is difficult to remove such compounds to an acceptable degree to purify the acetonitrile to a purity needed for recycling and/or recovery.

Importantly, it has been found that it is possible to utilize a substantially pure mixture of acetonitrile and toluene as a suitable wash solvent in the oligonucleotide synthesis process, rather than using pure acetonitrile. Therefore, it has been identified that there is a need to purify the waste stream to a substantially pure mixture of acetonitrile and toluene (i.e., removing the other impurities, such as iodine, thiols, and alkyl amines) in order to reuse the otherwise unusable contaminated acetonitrile waste stream as a wash solvent in the synthesis process. Furthermore, it has also been surprisingly found that, at certain compositions, the use of the purified mixture of acetonitrile and toluene as a wash solvent in the oligonucleotide synthesis process yields higher purity oligonucleotides as compared to a pure acetonitrile wash solvent. Therefore, the recycling of the purified waste stream results not only in a substantial decrease in wasted acetonitrile-based wash solvent (e.g., since the wash solvent is recycled back into the oligonucleotide synthesis process rather than being discarded), but the use of the purified waste stream also results in higher purity oligonucleotides.

As described relating to U.S. Pat. No. 10,336,690, the disclosure for which is expressly incorporated herein, removal of contaminates from the waste stream can be accomplished via fractional distillation in combination with contact with one or more absorbents/adsorbents. In this case, the waste stream can be fed to a fractionation/distillation column, where, during fractionation/distillation, the overhead fraction (distillate) includes the acetonitrile and the lower boiling point and/or volatile compounds (e.g., toluene, some water, and the vast majority of the iodine, thiols, and alkyl amines), while the bottom fraction includes the higher boiling point components of the waste stream. Also as described relating to U.S. Pat. No. 10,336,690, the condensed overhead stream can be sent to a series of absorbers, where, when contacted with different absorbents, a purified stream containing acetonitrile can be obtained. However, in this case, removal of the impurities consumes a substantial amount of absorbent due to the relatively high concentration of contaminants in the overhead fraction. As such, it has been identified that it may be advantageous to remove one or more of these contaminants prior to contacting the absorbers. This can lower the cost of the purification process, since both the size of the column and/or the amount of absorbent/adsorbent utilized can be reduced.

As used herein the terms “purifying” and variants thereof refer to the reduction of the amount of one or more impurities in a composition. As a general concept, purification may be accomplished via any number of techniques known in the art, depending on the nature of the composition to be purified and impurity to be reduced. Purification does not necessarily result in a pure product, completely absent of all impurities or of any particular impurity. Additionally, some of the methods and systems described herein utilize a plurality of purification steps, including fractionation of an acetonitrile-containing waste stream. In some embodiments, fractionation is an initial purification step.

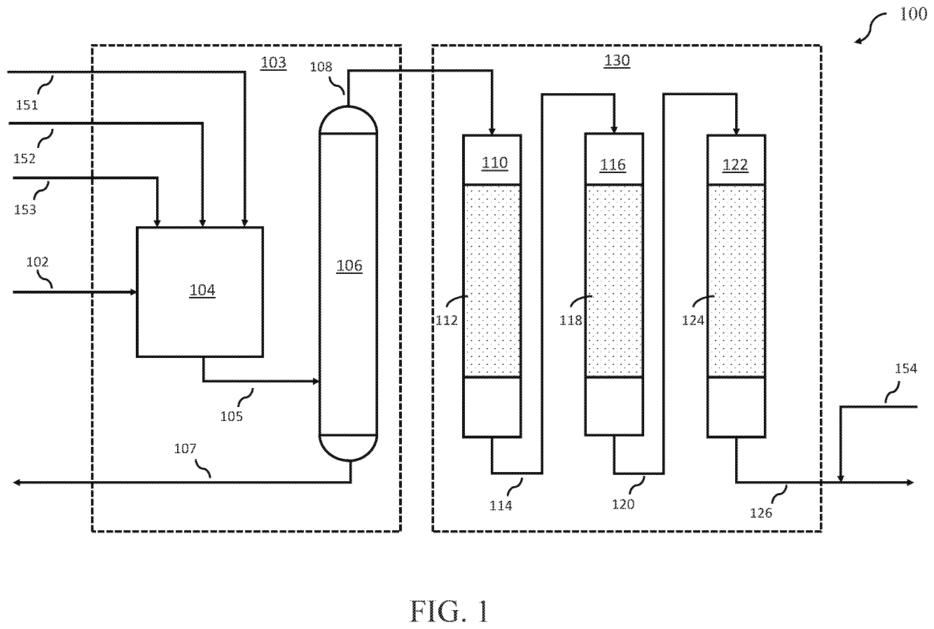

Referring to FIG. 1, in an exemplary embodiment, fractionation of a waste stream 102 is conducted using a system 100 having a fractionation zone 103 including a distillation or fractionation column 106. In specific embodiments, the fractionation/distillation column 106 contains internals such as packing, trays, sieves, bubble caps or similar mechanical configurations that can provide stages of multiple, step wise contact for vapor-liquid streams flowing through the column. The number of stages and types of internals will vary depending on the specific composition of the acetonitrile-containing waste stream, inlet location, reflux ratios, desired column efficiency, etc. As such, the internal profile of fractionation/distillation column 106 may vary from one application to another.

The waste stream 102 may come from various sources and contain some impurities with boiling points above and some impurities with boiling points below that of acetonitrile. In this regard, some waste stream purification methods rely on taking a “heart cut” of the distillate where two fractionation columns are utilized to firstly separate acetonitrile and lower boiling impurities (in a first overhead fraction) from higher boiling impurities (in a first bottoms fraction), and a second fractionation column to separate acetonitrile and other similarly boiling species (in a second bottoms fraction) from the lower boiling impurities (in a second overhead fraction).

However, as described herein, such complex fractionation by two columns is not required. Rather, in some embodiments, the fractionation zone 103 includes a single column 106, where a single overhead fraction 108 is collected up to a single cut point (e.g., just above the boiling point of acetonitrile or an acetonitrile-toluene mixture, such as about 81°-82° C. at standard pressure (1 ATM)).

As used herein, the term “overhead” fraction means a fraction removed from an upper portion of a fractionation column. The overhead fraction includes components of the original mixture with lower boiling points than components found in the “bottoms” (i.e., a fraction that remains at and/or is removed from a lower portion of the fractionation column). This overhead fraction 108 comprises acetonitrile and lower temperature boiling impurities. In some embodiments, the overhead fraction 108 may also comprise some higher boiling impurities, such as toluene due to azeotropic behavior with acetonitrile, as well as iodine.

Methods and systems described herein accomplish further purification of the overhead fraction 108 by condensing the overhead fraction 108 and contacting the condensed overhead fraction 108 with one or more adsorbents (e.g., adsorbents 112, 118, and 124 contained within columns 110, 116, and 122, respectively). The nature of specific adsorbents used in the subsequent purification steps is determined by the identity and levels of impurities which remain in the overhead fraction 108, and thus is impacted by the source of the waste stream 102.

As discussed above, numerous scientific and manufacturing processes utilize acetonitrile and toluene streams as a solvent or as a wash solution, and thus generate an acetonitrile waste stream. Various processes require differing degrees of purity of the acetonitrile and toluene stream, and/or may require certain degrees of purity with respect to one or more particular impurities.

One such process that generates a particularly large volume of acetonitrile waste is the manufacture of synthetic oligonucleotides (DNA and RNA). Oligonucleotide synthesis is generally conducted via a four step cycle (deblocking, activation/coupling, capping, and oxidation) with a pure acetonitrile wash between each cycle. As each step in a cycle uses different reagents, the acetonitrile waste stream generated from washing after each step is likely contaminated with different types and levels of impurities.

For instance, each oligonucleotide synthesis cycle typically begins with a blocked nucleotide covalently linked to a support. The blocked nucleotide is deblocked in a first step, typically via a reaction with an acidic solution, such as 3% trichloroacetic acid (TCA) or 3% dichloroacetic acid (DCA), in the presence of toluene or dichloromethane. Once the deblocking reaction is complete, the reagents are drained, and the deblocked oligonucleotides (attached to a support) are washed with an acetonitrile-based wash solvent. Impurities found in the resulting acetonitrile waste stream may include unreacted reagents, residual solvent, and/or byproducts of the deblocking reaction. For instance, impurities in the deblocking acetonitrile waste stream may include toluene, acetic acid, trichloromethane, and/or propanenitrile. Impurities in the deblocking acetonitrile waste stream typically constitute 0.1-30 wt. % of waste stream 102. In an exemplary embodiment, the waste stream 102 includes acetonitrile waste generated during an acetonitrile washing step after a deblocking step of an oligonucleotide synthesis.

The second step in the oligonucleotide synthesis cycle involves activating and coupling the deblocked oligonucleotides. Activation is carried out via contact with any suitable activator as known in the art. After activation, the support-bound material is treated with iodine and water in the presence of a weak base (e.g., pyridine, lutidine, or collidine) which oxidizes the phosphite triester into a tetracoordinated phosphate triester. The deblocked oligonucleotides are coupled (i.e., reacted), with a phosphoramidite to form a phosphite oligomer. Phosphoramidites are variously selected from among all available phosphoramidites, with the reactions proceeding as known in the art.

After coupling, the reagents are drained and pushed through the column by, and the solid support is washed by, an acetonitrile-based wash solvent. Again, impurities found in the resulting acetonitrile waste stream may include unreacted reagents, residual solvent, or byproducts of the activation and coupling reactions. For instance, impurities in the activation/coupling acetonitrile waste stream may include activator molecules, phosphoramidites, iodine, pyridine, propanenitrile, and/or toluene. Impurities in the coupling acetonitrile waste stream again typically constitute 0.1-30 wt. % of waste stream 102. In an exemplary embodiment, the waste stream 102 includes acetonitrile waste generated during an acetonitrile washing step after an activation/coupling step of an oligonucleotide synthesis.

Typically, during the coupling step, only a portion of the deblocked nucleotides react with the phosphoramidite, and any unreacted nucleotides must be capped. Capping is carried out by contacting the unreacted nucleotides with an amount of acetic anhydride and an amount of N-methylimidazole. After coupling, the reagents are drained/pushed through the column by, and the growing oligonucleotides on the solid support are washed by, an acetonitrile-based wash solvent. Again, impurities found in the resulting acetonitrile waste stream may include unreacted reagents, residual solvent, or byproducts of the capping reaction. For instance, impurities in the capping acetonitrile waste stream may include N-methylimidazole, acetic acid, acetic anhydride, lutidine, pyridine, tetrahydrofuran, propanenitrile, and/or toluene. Impurities in the capping acetonitrile waste stream again typically constitute 0.1-30 wt. % of waste stream 102. In an exemplary embodiment, the waste stream 102 includes acetonitrile waste generated during an acetonitrile washing step after a capping step of an oligonucleotide synthesis.

Finally, phosphite oligomers formed in the activation/coupling step are oxidized or sulfurized. Oxidation is accomplished, (e.g., via a reaction with) iodine in the presence of water and pyridine. Sulfurization is accomplished with a reaction of Phenyl acetyl disulfide or a similar chemical entity. After the oxidation or sulfurization, the reagents are drained/pushed through the column with the wash acetonitrile solution. The solid support containing the growing oligonucleotides is again washed with an acetonitrile wash stream. Impurities found in the resulting acetonitrile waste stream may include unreacted reagents, residual solvent, or byproducts of the oxidation reaction. For instance, impurities in an oxidation acetonitrile waste stream may include organic and inorganic iodine, toluene, tetrahydrofuran, pyridine, propanenitrile, and water. In an exemplary embodiment, the waste stream 102 includes acetonitrile waste generated during an acetonitrile washing step after an oxidation step of an oligonucleotide synthesis.

As will be appreciated, the type and amounts of impurities found in wash streams from each of the above describe steps can be different. The methods and systems described herein may be used to purify a waste stream 102 from a washing step after any particular step, or may be used to purify waste stream 102 that has been collected and pooled (e.g., within collection tank 104) from washing steps as described relating to deblocking, coupling, capping, and/or oxidation as described previously.

The identity and amount of impurities in the overhead fraction 108 may vary depending on the source of the waste stream 102 and appropriate adsorbent(s) may be selected on these compositions as well as the final grade (i.e., overall purity) of the acetonitrile/toluene end product desired.

For instance, an overhead fraction obtained by distillation of collected waste resulting from oligonucleotide synthesis may contain species such as water and toluene, as well as various other impurities at concentrations up to 30 weight percent of the waste stream. Such impurities may be organic and/or inorganic iodine containing compounds, sulfur containing compounds such as thiols (e.g., ethylthiotetrazole (ETT), phenyl acetyl disulfide (PADS)), basic nitrogen-containing compounds (e.g., pyridine, lutidine, picoline, 1-Methylimidazole), various alcohols (e.g., methanol), esters (e.g., acetic acid, dichloroacetic acid), nitriles (e.g., propanenitrile), and/or halogenated hydrocarbons (e.g., trichloromethane/dichloromethane).

FIG. 1 illustrates an exemplary embodiment of a system 100 used to purify a waste stream 102 received from an oligonucleotide syntheses process to a purity acceptable for recycling. In this case, the purification occurs via a system 100 including a fractionation zone 103, which includes a collection tank 104 and a fractionation/distillation column 106, as well as an adsorbent zone 130, which includes a first absorption column 110, a second absorption column 116, and a third absorption column 122.

As illustrated in FIG. 1, fractionation zone 103 includes collection tank 104 and fractionation/distillation column 106. In this case, waste stream 102 passes into fractionation zone 103 and is collected in collection tank 104. Collection tank 104 may be used to combine waste stream 102 into a pool, such that the various forms of waste stream 102 (e.g., as described above relating to the different compositions of deblocking, coupling, capping, and/or oxidation steps) are held in a common volume within collection tank 104.

Once waste stream 102 enters collection tank 104, it may be contacted with one or more reactive compounds (i.e., reducing compounds, scavengers, etc.) whereas some of the impurities within waste stream 102 react with, and are subsequently, precipitate from, waste stream 102.

For example, a first reactive compound 151 may be added to collection tank 104 and react with some of the components of waste stream 102. In this case, first reactive compound 151 may be an iodine reactive compound, which reacts with (e.g., reduces) one or more iodine containing compounds within waste stream 102. For example, the iodine reactive compound may be sodium thiosulfate, whereas the sodium thiosulfate anion reacts with the iodine of the iodine containing compounds, therefore reducing the iodine and subsequently precipitating iodine from waste stream 102, as illustrated in the representative reaction below.

I₂+2Na₂S₂O₃→2NaI+Na₂S₄O₆

In a second example, a second reactive compound 152 may be added to collection tank 104 and react with some of the components of waste stream 102. In this case, second reactive compound 152 may be a sulfur reactive compound, which reacts with (e.g., reduces) one or more sulfur containing compounds such as a thiol (e.g., ETT, PADS) within waste stream 102. For example, the sulfur reactive compound may be silver nitrate which reacts with the sulfur containing compounds, therefore reducing the sulfur containing compounds and subsequently precipitating the sulfur containing compounds from waste stream 102, as illustrated in the representative reaction below.

2RS—SR+2H₂O+AgNO₃→3RS—Ag+RSO₂H+2HNO₃

In a third example, a third reactive compound 153 may be added to collection tank 104 and react with some of the components of waste stream 102. In this case, third reactive compound 153 may be an acidic reactive compound, which reacts with (e.g., reduces) one or more basic nitrogen containing compounds such as alkyl amines (e.g., pyridine, lutidine, picoline, 1-Methylimidazole) within waste stream 102. In this case, the acidic reactive compound may be a carboxylic acid (e.g., formic acid) which reacts with (e.g., reduces) the basic nitrogen containing compounds, and subsequently precipitates the basic nitrogen containing compounds from the waste stream 102, as illustrated in the representative reaction below.

R1,R2-N—H+HCOOH→R1-R2-N—COH+H₂O

In some cases, two or more of the reactive compounds 151, 152, and/or 153 may be added to waste stream 102 within collection tank 104 at the same, or subsequent times, and each of the reactive compounds 151, 152, and/or 153 may react combinationally with compounds within waste stream 102.

For example, the iodine reactive compound 151 can be added at the same time, prior to, or subsequent to, the sulfur reactive compound 152, and both the iodine reactive compound 151 and the sulfur reactive compound 152 react with the iodine containing compounds and the sulfur containing compounds within waste stream 102 respectively. In this case, the sulfur reactive compound 152 may further react with the reduced iodine containing compounds to further precipitate the iodine from waste stream 102. For example, in the case where the iodine reactive compound 151 is sodium thiosulfate, and the sulfur reactive compound 152 is silver nitrate, the silver nitrate may further reduce the iodide produced via the reaction of the sodium thiosulfate and the volatile iodine, and enhance the recovery (e.g., precipitation) of the iodine from waste stream 102. However, the addition of silver nitrate to reduce and precipitate iodide is not required, and sufficient reduction and precipitation of iodine without the use of silver nitrate is possible.

In another example, the iodine reactive compound 151 can be added at the same time, prior to, or subsequent to, the acidic reactive compound 153, and both the iodine reactive compound 151 and acidic reactive compound 153 react with the iodine containing compounds and the basic nitrogen containing compounds within waste stream 102 respectively. In this case the chemical interaction between iodine reactive compound 151 and acidic reactive compound 153, as well as the intermediates formed when reducing the iodine containing compounds and the basic nitrogen containing compounds, may act in conjunction to reduce the sulfur containing compounds of waste stream 102. For example, in the case where the iodine reactive compound 151 is sodium thiosulfate, and the acidic reactive compound 153 is formic acid, the interactions of the sodium thiosulfate and formic acid with each of the iodine containing compounds and the basic nitrogen containing compounds can create a matrix effect, whereas the intermediates formed by these reactions react with (e.g., reduce) the sulfur containing compounds (e.g., thiols) in the waste stream 102. In this regard, it may be possible to achieve a desired reduction of all three impurities (e.g., iodine containing compounds, sulfur containing compounds, and basic nitrogen containing compounds) with the use of iodine reactive compound 151 and the acidic reactive compound 153 (e.g., reducing the sulfur containing compounds without the use of sulfur reactive compounds 152).

In yet another example, all three of the reactive compounds 151, 152, and/or 153 may be added to waste stream 102 within collection tank 104, and each of the reactive compounds 151, 152, and/or 153 may react at the same time, and/or in combination, to reduce each of the iodine containing compounds, sulfur containing compounds, and basic nitrogen containing compounds within waste stream 102. In this case, each of the iodine containing compounds, sulfur containing compounds, and basic nitrogen containing compounds may be precipitated from waste stream 102 by the reactions with reactive compounds 151, 152, and/or 153, as well as the intermediates that are produced by such reactions.

Although FIG. 1. illustrates each of the reactive compounds 151, 152, and/or 153 as being added separately to collection tank 104 (i.e., by different feed lines), it is possible to add two or more reactive compounds in combination. For example, reactive compounds 151 and 152 could be added to collection tank 104 by the same feed line, rather than by separate feed lines, as illustrated in FIG. 1. The foregoing also applies to any combination of reactive compounds 151, 152, and 153 where each of the components can be added individually, or in any combination, including all three reactive compounds 151, 152, and 153 added to collection tank 104 by the same feed line. Alternatively, in some embodiments, any one of reactive compounds 151, 152 and/or 153 could be added to feed stream 105, rather than to collection tank 104. In this case, it may be possible to remove collection tank 104 from system 100, and dose each of the reactive compounds 151, 152, and/or 153 into waste stream 102 (e.g., removing collection tank 104 from fractionation zone 103, where waste stream 102 is in-lined does with each of reactive compounds 151, 152, and/or 153, and at the point in waste stream 102 whereas the reactive compound(s) are added, waste stream 102 transitions to feed stream 105.)

Each of waste stream 102 and reactive compounds 151, 152, and/or 153, are collected within collection tank 104, and fed as feed stream 105 to fractionation/distillation column 106. Fractionation/distillation column 106 is used to separate/fractionate the components of feed stream 105 into an overhead fraction 108 and a bottom fraction 107.

Overhead fraction 108 contains at least the majority of acetonitrile originally present in waste stream 102. For example, overhead fraction 108 may contain 75% or more, such as 85% or more, such as 95% or more, of the acetonitrile originally contained in the waste stream 102. That is, the overhead fraction 108 may contain about 75% to about 100%, such as about 85% to about 100%, such as about 95% to about 100%, of the acetonitrile originally contained in the waste stream 102.

In addition to the acetonitrile, overhead fraction 108 may also contain water, toluene, and other lower boiling point impurities. For example, in the case where approximately 1 weight percent (e.g., 10,000 ppm) of water is present in waste stream 102, the overhead fraction 108 may contain as much as 10,000 ppm, however more preferably 100 ppm, and more preferably 50 ppm and more preferably 30 ppm of water. In this case, water may form an azeotrope with one of more components of the overhead fraction 108, such as an azeotrope formed with acetonitrile. In this case, and as will be further described herein, water can be removed from the overhead fraction 108 by a variety of methods, including contact with a desiccant.

An “azeotrope” (or “azeotropic”) composition is a unique combination of two or more components. An azeotrope can be either homogenous (which has one liquid phase) or heterogenous (which has two liquid phases). An azeotrope composition can be characterized in various ways. For example, at a given pressure, an azeotrope composition boils at a constant characteristic temperature which is either greater than the higher boiling point component (maximum boiling azeotrope) or less than the lower boiling point component (minimum boiling azeotrope). However, in the case of a heterogenous azeotrope the boiling point of the azeotrope will always be below the boiling point of the lower boiling point component. In the case of a heterogenous azeotrope then at this characteristic temperature the composition of each of the two liquid phases and the vapor phase will remain constant upon boiling. The azeotrope composition does not fractionate upon boiling or evaporation. Therefore, the components of the azeotrope composition cannot be separated during a phase change.

An azeotrope composition is also characterized in that at the characteristic azeotrope temperature, the bubble point pressure of the liquid phase is identical to the dew point pressure of the vapor phase.

The behavior of an azeotrope composition is in contrast with that of a non-azeotrope composition in which during boiling or evaporation, the liquid composition changes to a substantial degree.

For the purposes of the present disclosure, an azeotrope composition is characterized as that composition which boils at a constant characteristic temperature, the temperature being lower (a minimum boiling azeotrope) than the boiling points of the two or more components, and thereby having the same composition in both the vapor and liquid phases.

One of ordinary skill in the art would understand however that at different pressures, both the composition and the boiling point of the azeotrope composition will vary to some extent. Therefore, depending on the temperature and/or pressure, an azeotrope composition can have a variable composition. The skilled person would therefore understand that composition ranges, rather than fixed compositions, can be used to define azeotrope compositions. In addition, an azeotrope may be defined in terms of exact weight percentages of each component of the compositions characterized by a fixed boiling point at a specified pressure.

Azeotrope or azeotrope-like compositions can be identified using a number of different methods.

For the purposes of this disclosure the azeotrope or azeotrope-like composition is identified experimentally using an ebulliometer (Walas, Phase Equilibria in Chemical Engineering, Butterworth-Heinemann, 1985, 533-544). An ebulliometer is designed to provide extremely accurate measurements of the boiling points of liquids by measuring the temperature of the vapor-liquid equilibrium.

The boiling points of each of the components alone are measured at a constant pressure. As the skilled person will appreciate, for a binary azeotrope or azeotrope-like composition, the boiling point of one of the components of the composition is initially measured. The second component of the composition is then added in varying amounts and the boiling point of each of the obtained compositions is measured using the ebulliometer at said constant pressure. In the case of a ternary azeotrope the initial composition would comprise of a binary blend and a third component is added in varying amounts. The boiling point of each of the obtained ternary compositions is measured using the ebulliometer at said constant pressure.

The measured boiling points are plotted against the composition of the tested composition, for example, for a binary azeotrope, the amount of the second component added to the composition, (expressed as either weight % or mole %). The presence of an azeotrope composition can be identified by the observation of a maximum or minimum boiling temperature which is greater or less than the boiling points of any of the components alone.

As the skilled person will appreciate, the identification of the azeotrope or azeotrope-like composition is made by the comparison of the change in the boiling point of the composition on addition of the second component to the first component, relative to the boiling point of the first component. Thus, it is not necessary that the system be calibrated to the reported boiling point of the particular components in order to measure the change in boiling point.

In the case where overhead fraction 108 contains toluene, the amount of toluene present in overhead fraction 108 may be based upon either an azeotropic interaction between acetonitrile and toluene or the composition of toluene present in feed stream 105. For example, in the case of the azeotropic interaction between acetonitrile and toluene, the relative weight percentages of acetonitrile and toluene of the overhead fraction 108 may depend upon the operating temperature and pressure of the fractionation/distillation column 106. For example, in the case where the fractionation/distillation column 106 is operating at a temperature between 80° C. and 86° C., and a pressure of 1 ATM, the azeotropic composition comprises approximately 76 weight percent acetonitrile and 24 weight percent toluene. In a second example, the relative weight percent of toluene in feed stream 105 may be less than the relative weight percent of toluene of the acetonitrile and toluene azeotrope (e.g., a relative weight percent of toluene of feed stream 105 being less than 24 weight percent). In this case, overhead fraction 108 may contain substantially the same relative weight percents of acetonitrile and toluene of feed stream 105 For example, in the case where feed stream 105 contains approximately 80 relative weight percent acetonitrile and 20 relative weight percent toluene, overhead fraction 108 also contains the 80 relative weight percent acetonitrile and 20 relative weight percent toluene (e.g., the relative weight percent of toluene not increasing to the azeotropic composition of 24 relative weight percent toluene). As such, the relative weight percent of toluene in overhead fraction 108 may, at maximum, be based upon the azeotropic interaction between acetonitrile and toluene at the operating temperature and pressure of fractionation/distillation column 106, or may be less than the azeotropic composition when feed stream 105 contains a lower relative weight percent of toluene than the azeotropic composition. Said another way, the relative weight percent of acetonitrile to toluene may, at minimum, be based upon the azeotropic composition of acetonitrile and toluene, or, may be a higher relative weight percent than the azeotropic composition when feed stream 105 contains less toluene than the azeotropic composition.

However, it is also possible to operate fractionation/distillation column 106 at a variety of temperatures and pressures to achieve a desired recovery of acetonitrile whereas the relative compositions of acetonitrile and toluene may change. Additionally, although illustrated as single distillation column 106, and in the case of pressure swing distillation, more than one column 106 may be used (e.g., two columns 106 in series operating at different temperatures and pressures). Therefore, in this regard, although described as relating to an azeotropic composition of 76 weight percent acetonitrile and 24 weight percent toluene, other azeotropic compositions of acetonitrile and toluene may be possible.

In addition to the acetonitrile and toluene, the overhead fraction 108 may contain one or more impurities, such as iodine, sulfur containing compounds such as thiols (e.g., ethylthiotetrazole (ETT), BMT, phenyl acetyl disulfide (PADS)), basic nitrogen-containing compounds (e.g., pyridine, lutidine, picoline, N-Methylimidazole), and esters (e.g., acetic acid, dichloroacetic acid), each of which may be carried over in the overhead fraction 108 due to the compounds boiling points (e.g., lower boiling point than the operating temperature of fractionation/distillation column 106) and/or due to the chemical interactions of compounds within waste stream 102 (i.e., as is the case of iodine and water). However, as described previously, one or more reactive compounds 151, 152, and/or 153 may be added to waste stream 102, such that when feed stream 105 containing such reactive compounds is fractionated, the overhead fraction 108 includes substantially less of these impurities.

For example, in the case where reactive compound 151 is added to waste stream 102 in collection tank 104, and first reactive compound 151 is an iodine reactive compound (e.g., sodium thiosulfate), the reaction (e.g., reduction) of the iodine containing compounds of waste stream 102 via sodium thiosulfate substantially reduces the amount of iodine present in the overhead fraction 108. In this case, rather than the iodine remaining in a volatile state (e.g., that when distilled, would otherwise be present in overhead fraction 108), the iodine is precipitated from feed stream 105 within distillation column 106, and the overhead fraction 108 contains a relatively low amount of iodine. For example, in the case where 5 ppm of iodine is present in feed stream 105, first reactive compound 151 may reduce the amount of iodine in the overhead fraction 108 to less than 2 ppm, and in some cases, less than 1 ppm, and more preferably less than 0.5 ppm, and in some cases, to trace amounts (e.g., substantially free from iodine). In these examples, the amount of iodine present in the overhead fraction 108 is substantially lower than if first reactive compound 151 was not added to waste stream 102 prior to fractionation/distillation.

In a second example, second reactive compound 152 is a sulfur reactive compound (e.g., silver nitrate) that is added to waste stream 102 in collection tank 104. In this example, the reaction (e.g., reduction) of the sulfur containing compounds (e.g., thiols) of waste stream 102 via sodium thiosulfate substantially reduces the amount of the thiols present in the overhead fraction 108. In this case, rather than the thiols remaining in a volatile state (e.g., that when distilled, would otherwise be present in overhead fraction 108), the thiols are precipitated from feed stream 105 within distillation column 106, and the overhead fraction 108 contains a relatively low amount of the sulfur containing components. For example, in the case where 200 ppm of a thiol compound is present in waste stream 102, second reactive compound 152 may reduce the amount of the thiol compound in the overhead fraction to less than 10 ppm, and in some cases, less than 5 ppm, and more preferably, less than 1 ppm, and in some cases, less than 0.5 ppm, and in still further cases, to trace amounts (e.g., substantially free from thiols). In these examples, the amount of thiols present in the overhead fraction 108 are substantially lower than if second reactive compound 152 was not added to waste stream 102 prior to fractionation/distillation.

In a third example, third reactive compound 153 is an acidic reactive compound (e.g., formic acid) that is added to waste stream 102 in collection tank 104. In this example, the reaction (e.g., reduction) of the basic nitrogen containing compounds (e.g., alkylamines) of waste stream 102 via formic acid substantially reduces the amount of the alkylamines present in the overhead fraction 108. In this case, rather than the alkylamines remaining in a volatile state (e.g., that when distilled, would otherwise be present in overhead fraction 108), the alkylamines are precipitated from feed stream 105 within distillation column 106, and the overhead fraction 108 contains a relatively low amount of the basic nitrogen containing compounds. For example, in the case where up to 5 weight percent (e.g., 50000 ppm) of an alkylamine compound is present in waste stream 102, third reactive compound 152 may reduce the amount of the basic nitrogen containing compound in the overhead fraction to less than 500 ppm, and in some cases, less than 200 ppm, and more preferably, less than 100 ppm. In other examples, the concentration of the alkylamine compound may be substantially lower in waste stream 102, such as approximately 100 ppm. In this case, the third reactive compound 152 may reduce the amount of the alkylamine compound in the overhead fraction to less than 10 ppm, and in some cases, less than 5 ppm, and more preferably, less than 1 ppm, and in some cases, less than 0.5 ppm, and in still further cases, to trace amounts (e.g., substantially free from alkylamines). In these examples, the amount of alkylamine compounds present in the overhead fraction 108 are substantially lower than if third reactive compound 153 was not added to waste stream 102 prior to fractionation/distillation.

In each of the cases referenced above, the bottom fraction 107 includes the lower boiling point compounds (e.g., water), as well as any of the precipitated compounds such as iodine, sulfur containing compounds, and/or basic nitrogen containing compounds. In this case, the bottom fraction 107 is recovered and may be further processed to remove some of the compounds contained in bottom fraction 107. For example, in the case where silver nitrate is used either as a reductive agent for the sulfur containing compounds and/or the iodine, the silver may be recovered via further processing of bottom fraction 107.

In an exemplary embodiment, condensed overhead fraction 108 exits the fractionation zone 103 and is passed to an adsorbent zone 130. In the adsorbent zone 130, the condensed overhead fraction 108 enters a first adsorbent column 110 that is configured to contain a first adsorbent 112. The first overhead fraction 108 is purified by contact with the first adsorbent 112 to generate purified acetonitrile and toluene stream 114.

As described previously, the amount of iodine containing species present in the overhead fraction 108 are substantially reduced by first reactive compound 151, however, some iodine containing components may still be present in overhead fraction 108. In this case, overhead fraction 108 may be contacted with an adsorbent 112 selected to reduce the amounts of these organic and/or inorganic iodine-containing species, resulting in a purified acetonitrile and toluene stream 114. Suitable adsorbents include silver (Ag)-exchange zeolites. In some embodiments, the Ag-exchange zeolites are Ag-exchange faujasite zeolites, and in particular, faujasite zeolites with a silicon:aluminum (Si:Al) mole ratio of at least about 1.2, such as with a Si:Al mole ratio of at least about 2.0. In some embodiments utilizing Ag-exchange faujasite zeolites, Ag is present at a minimum of about 3 wt. %, such as at least about 15 wt. %. In some specific embodiments, the Ag is partially ion-exchanged and partially precipitated on the zeolite, with the fraction of Ag ion-exchanged depending on the Si:Al mole ratio and wt. % Ag. Typically, however, at least about 10 wt. % of the Ag is ion-exchanged on the zeolite. This is not to say that iodine-reducing adsorbents are limited to faujasite zeolites. Rather, any other suitable zeolite may be used.

In embodiments utilizing one or more Ag-exchange faujasite zeolites to reduce organic and/or inorganic iodine containing species and sulfur containing species present in the condensed overhead fraction, the adsorbent(s) may be used by contacting the condensed overhead fraction 108 with the adsorbent(s) at a weight ratio of at least about 1:100,000 adsorbent:condensed overhead fraction 108. In some embodiments, the weight ratio is at least about 1:10,000, 1:1000, or 1:100 adsorbent:condensed overhead fraction 108. Contact may be conducted via a batch process or via a continuous process with suitable apparatus, temperatures, pressures, and flow rates.

In some embodiments, the methods provided herein are used to purify a waste stream 102 comprising organic and/or inorganic iodine-containing compounds whereas the resulting purified acetonitrile and toluene stream 114 comprises about 25 ppm of iodine or less, such as about 10 ppm of iodine or less, such as about 5 ppm of iodine or less, such as about 1 ppm of iodine or less, such as about 0.5 ppm of iodine or less, and in some cases, substantially free from iodine. In some cases, In some embodiments, the resulting purified acetonitrile and toluene stream 114 comprises about 0.1 ppm to about 25 ppm of iodine, such as about 0.1 ppm to about 10 ppm of iodine, such as about 0.1 ppm to about 5 ppm of iodine, such as about 0.1 ppm to about 1 ppm of iodine, such as about 0.1 ppm to about 0.5 ppm of iodine.

In some embodiments, the methods provided herein are used to purify a waste stream 102 comprising organic and/or inorganic sulfur-containing compounds as impurities. As described previously, the amount of sulfur containing compounds present in the overhead fraction 108 are substantially reduced by second reactive compound 152, however, some sulfur containing components may still be present in overhead fraction 108. In this case, overhead fraction 108 may be contacted with an adsorbent 112 selected to reduce the amounts of these sulfur containing compounds, resulting in a purified acetonitrile and toluene stream 114. In some embodiments, the resulting purified acetonitrile and toluene stream 114 comprises about 10 ppm of sulfur or less, such as about 6 ppm of sulfur or less, such as about 5 ppm of sulfur or less, such as about 4 ppm of sulfur or less. In some embodiments, the resulting purified acetonitrile and toluene stream 114 comprises from about 0.5 ppm to about 10 ppm of sulfur, such as from about 0.5 ppm to about 6 ppm of sulfur, such as from about 0.5 ppm to about 5 ppm of sulfur, such as about 0.5 ppm to about 4 ppm of sulfur.

In some embodiments, the methods provided herein are used to purify a waste stream 102 comprising basic nitrogen containing compounds, such as pyridine, pyridine, lutidine, picoline, N-Methylimidazole. As described previously, the amount of basic nitrogen containing compounds present in the overhead fraction 108 are substantially reduced by third reactive compound 152, however, some of the basic nitrogen containing components may still be present in overhead fraction 108. In this case, the purified acetonitrile and toluene stream 114 is passed to a second adsorbent column 116 that is configured to contain abasic N reducing adsorbent 118. Again, a basic N reducing adsorbent may be the only adsorbent used; alternatively, a basic N reducing adsorbent may be one of a plurality of adsorbents used. If a plurality of adsorbents is used, the adsorbents may be used sequentially. In such embodiments, a basic N reducing adsorbent may be used at any position in the sequence.

In the particular exemplary embodiment in FIG. 1, the purified acetonitrile and toluene stream 114 is contacted with a basic N reducing adsorbent 118, resulting in purified acetonitrile and toluene stream 120 that comprises an amount of basic N-containing compound impurities that is reduced relative to waste stream 102. Purified acetonitrile and toluene stream 120 may be collected for use or subjected to further purification as desired.

In some embodiments, a basic N adsorbent may comprise an acidic cation exchange resin. In such embodiments, the relative proportion of adsorbents used per volume of the acetonitrile and toluene stream to be purified (e.g., condensed overhead fraction 108 or purified acetonitrile and toluene stream 114) may vary depending on the specific adsorbent, amount of basic N-containing impurities contained in the acetonitrile and toluene stream, and desired level of basic N-containing impurities in the resulting purified acetonitrile and toluene stream. In some embodiments, an acidic cation exchange resin such as Amberlyst™-15 is used to reduce basic N-containing impurities in an acetonitrile and toluene stream at a ratio of at least about 1 g adsorbent to 10 L acetonitrile and toluene stream, such as at least about 1 g adsorbent to 0.1 L acetonitrile and toluene stream.

Thus, in some embodiments the methods provided herein are used to purify a waste stream 102 comprising one or more basic N-containing compounds as impurities. In some embodiments, a purified acetonitrile and toluene stream is generated by removing such impurities from an acetonitrile and toluene stream such that the resulting purified acetonitrile and toluene stream comprises about 100 ppm of basic N-containing compounds or less, such as about 50 ppm of basic N-containing compounds or less, such as about 25 ppm of basic N-containing compounds or less, such as about 10 ppm of basic N-containing compounds or less. In some embodiments, a purified acetonitrile and toluene stream 120 is generated by removing such impurities from an acetonitrile and toluene stream such that the resulting purified acetonitrile and toluene stream comprises from about 1 ppm to about 100 ppm of basic N-containing compounds, such as from about 1 ppm to about 50 ppm of basic N-containing compounds, such as from about 1 ppm to about 25 ppm of basic N-containing compounds, such as from about 1 ppm to about 10 ppm of basic N-containing compounds.

In some embodiments, an acid exchange resin may also reduce the amount of cations present in the purified acetonitrile and toluene stream 114. For example, impurities such as cationic Fe, Mg, Cr, Ni, Ag, and I₂, may be reduced by contact with a cation exchange resin. In some embodiments, cation reduction occurs concurrently with basic N reduction via contact with a basic N reducing adsorbent comprising an acidic cation exchange resin as described above. In some embodiments, the purified acetonitrile and toluene stream 120 comprises less than about 5 ppm cationic Fe, such as from about 0.1 ppm to about 5 ppm, as an impurity. In some embodiments, the purified acetonitrile and toluene stream 120 comprises less than about 5 ppm cationic Mg, such as from about 0.1 ppm to about 5 ppm, as an impurity. In some embodiments, the purified acetonitrile and toluene stream 120 comprises less than about 5 ppm cationic Cr, such as from about 0.1 ppm to about 5 ppm, as an impurity. In some embodiments, the purified acetonitrile and toluene stream 120 comprises less than about 5 ppm cationic Ni, such as from about 0.1 ppm to about 5 ppm, as an impurity. In some embodiments, the purified acetonitrile and toluene stream 120 comprises less than about 5 ppm cationic Ag, such as from about 0.1 ppm to about 5 ppm, as an impurity. In some embodiments, the purified acetonitrile and toluene stream 120 comprises less than about 5 ppm iodine/iodide, such as from about 0.1 ppm to about 5 ppm, as an impurity.

It may be desirable to reduce the amount of water present in the condensed purified acetonitrile and toluene stream 120. In some embodiments, including the example shown in FIG. 1, the purified acetonitrile and toluene stream 120 passes to a third adsorbent column 122. In these embodiments, the third adsorbent column 122 is configured to contain a water-reducing adsorbent (i.e., a desiccant) 124. The purified acetonitrile and toluene stream 120 is contacted with the water reducing adsorbent 124 to produce a purified acetonitrile and toluene mixture 126 that exits the adsorption zone 130.

In some embodiments, potassium (K)-exchanged Linde Type A (LTA)-type molecular sieves bound with an inorganic binder may be used as a water removal adsorbent 124. It has surprisingly been found that porous desiccants exhibit increased performance (i.e., higher water adsorption capacity) for reduction of water in acetonitrile waste streams if they have been treated to partially close their pore structure. Thus, in some embodiments, one or more porous desiccants (including one or more K-exchanged LTA-type molecular sieves bound with inorganic binders) may be treated by any suitable process as known in the art to partially close the pores of the desiccant prior to use. However, care must be taken not to close the pores too much and thus restrict water adsorption capacity. The degree of pore closure may be measured by determining the smallest molecule that will not adsorb more than 1 wt. % of the molecule when exposed at one atmosphere partial pressure, also known as a plug-gauge molecule. In some embodiments, the desiccant is an LTA-type adsorbent that has been treated with stream to partially close the desiccant's pore structure to the point that the desiccant has a limited ability to adsorb ethylene, difluoromethane, or both.

In embodiments utilizing one or more LTA-type adsorbents to reduce water in a purified acetonitrile and toluene stream 120 from waste stream 102, the adsorbent(s) may be used at a weight ratio of at least 5-10 mg desiccant per gram of acetonitrile to be purified. In some embodiments, one or more LTA-type adsorbents are used with a minimum water adsorption capacity of 10 wt. %. In embodiments utilizing one or more LTA-type adsorbents to reduce water in a purified acetonitrile and toluene stream 120, the adsorbent(s) may be used at a weight ratio of at least 5-10 mg desiccant per gram of acetonitrile to be purified. In some embodiments, one or more LTA-type adsorbents are used with a minimum water adsorption capacity of 10 wt. %. Contact may be conducted via a batch or a continuous process, with suitable apparatus, temperatures, pressures, and flow rates.

In some embodiments, reducing the amount of water present in the condensed purified acetonitrile and toluene stream 120 is conducted via a liquid phase process. In these embodiments, the purified acetonitrile and toluene stream 120 is in a liquid phase when it is contacted with the water-reducing adsorbent 124.

In the alternative or in addition, reducing the amount of water present in a purified acetonitrile may be conducted via a vapor phase process, such as vapor phase pressure-swing adsorption (PSA). In an exemplary embodiment, a purified acetonitrile and toluene stream leaving a fractionation column may be processed so as to reduce or eliminate condensation of the overhead fraction. Rather, a purified acetonitrile and toluene stream leaving a fractionation column is further heated, e.g., superheated by about 25° C. above the fractionation temperature, and the heated vapor is fed to a column containing a water-reducing adsorbent. In some embodiments, the water-reducing adsorbent is a potassium (K)-exchanged Linde Type A (LTA)-type 20 molecular sieves bound with an inorganic binder, as described above, which may or may not have partially closed pores. After contact with the water-reducing adsorbent, the vapor is directed to a heat exchanger where it is condensed into a liquid phase purified acetonitrile and toluene. This liquid phase purified acetonitrile and toluene mixture may be subjected to further purification, including via contact with other adsorbent materials as described herein, as desired. It will be appreciated by those of skill in the art that a plurality of columns containing water-reducing adsorbents may be utilized in parallel in the methods described herein such that after saturation of the adsorbent in a first column, the acetonitrile and toluene mixture vapor to be dried may be directed to a second column, while the adsorbent in the first column is regenerated. In some embodiments, regeneration conditions include stripping water from an adsorbent by reducing the pressure and condensing the released materials as a liquid waste stream.

In some embodiments, at least a portion of the liquid waste stream may be redirected back to the fractionation column for further processing. In some embodiments, the average water delta load (i.e., the change in average water content of the adsorbent from the beginning to the end of an adsorption step) is about 1 to about 3 kg of water per 100 kg of adsorbent. In these embodiments, the adsorption conditions, such as pressure, feed rate, and flow rate, may be such that 45 an adsorption step may take from about 5 to about 10 minutes.

Thus, in some embodiments the methods provided herein are used to purify a waste stream 102 comprising water as an impurity. In some related embodiments, the waste stream 102 is fractionated and the overhead fraction is contacted with a desiccant with a partially closed pore structure. In some embodiments, the purified acetonitrile and toluene mixture 126 that has been purified by contact with a desiccant comprises about 2000 ppm of water or less, such as about 1000 ppm of water or less, such as about 500 ppm of water or less, such as about 150 ppm of water or less, 100 ppm of water or less, such as about 50 ppm of water or less, such as about 25 ppm of water or less.

While the various embodiments above describe the waste stream passing from a fractionation column, then sequentially through an iodine-reducing adsorbent, through a basic N-containing compound-reducing adsorbent, and through a water-reducing adsorbent, it will be appreciated that the scope herein is not so limited. Rather, it will be understood that only one or two of the adsorbents can be used in addition to the fractionation column and/or that the adsorbents can be used in a different order, without limit For example, after fractionation and condensation, the condensed overhead fraction 108 can be contacted with the basic N-containing compound-reducing adsorbent and the resulting acetonitrile and toluene stream contacted with the water-reducing adsorbent, or the condensed overhead fraction 108 can be contacted with a water-reducing adsorbent and the resulting stream contacted with the iodine-reducing adsorbent.

In some embodiments, the methods provided herein are used to purify a waste stream 102 from oligonucleotide synthesis to generate a purified acetonitrile and toluene mixture 126 suitable for reuse in various industrial or manufacturing processes. In some embodiments, the purified acetonitrile and toluene mixture 126 is suitable for reuse as a between-step wash solution in an oligonucleotide synthesis conducted with between-step acetonitrile washes. In some embodiments, the purified acetonitrile and toluene mixture 126 is greater than about 85% pure, such as greater than or equal to about 95% pure, as determined by gas chromatography.

As described previously, it has been found that, in the case of oligonucleotide syntheses processes, the use of a blended solution of toluene and acetonitrile as a wash solvent has been found not only to be acceptable (e.g., yielding similar purities of the produced oligonucleotides, as compared to a pure acetonitrile wash solvent), but in some cases, has been surprisingly found to increase the purity of the oligonucleotides produced as compared to a pure acetonitrile wash solvent.

For instance, in a first set of cases, various wash solvents comprising different acetonitrile and toluene purities where tested, and resulting oligonucleotide purities were obtained as based upon a first type of support resin and first type of oligonucleotide test sequence (e.g., Test-20; 20-mer, etc.), and it was surprisingly found that blended solution of toluene and acetonitrile as a wash solvents increased the purity of the first oligonucleotide purity, as compared to a pure acetonitrile wash solvent

For example, in the case where pure acetonitrile is used as a wash solvent in the oligonucleotide synthesis process which utilizes the first type of support resin and a first oligonucleotide test sequence (Test-20), the purity of the resulting synthesized oligonucleotide, as determined by liquid chromatography, may be in the 70% to 73% range, such as in the 70.2% to 72.2% range.

However, in the case where a substantially pure mixture of 95 weight % acetonitrile and 5 weight % toluene is used as a wash solvent in the oligonucleotide synthesis process with the first type of support resin and test-20 oligonucleotide sequence, it has been surprisingly found that the resulting synthesized oligonucleotide is slightly more pure than a synthesized oligonucleotide produced using pure acetonitrile as a wash solvent. For example, the oligonucleotide purity obtained from an oligonucleotide synthesis process using the 95 wt. % acetonitrile/5 wt. % toluene blend may have a purity in the 70% to 74% purity range, and more particularly, in the 70.7% to 73.8% purity range. Therefore, it may be advantageous to utilize a blend of 95 wt. % acetonitrile/5 wt. % toluene as a wash solvent in the oligonucleotide synthesis process, as compared to pure acetonitrile.

In another example, a substantially pure mixture of 85 weight % acetonitrile and 15 weight % toluene is used as a wash solvent in the oligonucleotide synthesis process with that same first support resin and Test-20 oligonucleotide sequence. In this case, it has been surprisingly found that the resulting synthesized oligonucleotide is slightly more pure than a synthesized oligonucleotide produced using pure acetonitrile as a wash solvent. For example, the oligonucleotide purity obtained from an oligonucleotide synthesis process using the 85 wt. % acetonitrile/15 wt. % toluene blend may have purity in the 72% to 76% purity range, and more particularly, in the 72.5% to 76.4% purity range. Therefore, it may be advantageous to utilize a blend of 85 wt. % acetonitrile/15 wt. % toluene as a wash solvent in the oligonucleotide synthesis process, as compared to pure acetonitrile.

As yet another example, in the case where a substantially pure mixture of the azeotropic composition of 76 weight % acetonitrile and 24 weight % toluene is used as a wash solvent in the oligonucleotide synthesis process with the first solid support resin and Test-20 Oligonucleotide sequence, it has been surprisingly found that the resulting synthesized oligonucleotide is slightly more pure than a synthesized oligonucleotide produced using pure acetonitrile as a wash solvent. For example, the oligonucleotide purity obtained from an oligonucleotide synthesis process using the 76 wt. % acetonitrile/24 wt. % toluene blend may have a purity in the 71% to 73% purity range, and more particularly, in the 71.3% to 72.2% purity range. Therefore, it may be advantageous to utilize a blend of 76 wt. % acetonitrile/24 wt. % toluene as a wash solvent in the oligonucleotide synthesis process, as compared to pure acetonitrile.

In a second set of cases, various wash solvents comprising different acetonitrile and toluene purities where tested, and resulting oligonucleotide purities were obtained as based upon a second type of support resin and second type of oligonucleotide test sequence (e.g., Test-18; 18-mer), and it was surprisingly found that blended solution of toluene and acetonitrile as a wash solvents also increased the purity of the second oligonucleotide purity, as compared to a pure acetonitrile wash solvent.

For example, where the second support resin and the second oligonucleotide test sequence (Test-18) are used, and where the pure acetonitrile is used as a wash solvent in the oligonucleotide synthesis process, the purity of the resulting synthesized oligonucleotide, as determined by liquid chromatography, may be in the 91% to 93% range, such as in the 91.2% to 93.1% range.

However, with the same second support resin (second resin) and test Oligonucleotide test sequence (Test 18), where a substantially pure mixture of 90 weight % acetonitrile and 10 weight % toluene is used as a wash solvent in the oligonucleotide synthesis process, it has been surprisingly found that the resulting synthesized oligonucleotide is slightly more pure than a synthesized oligonucleotide produced using pure acetonitrile as a wash solvent. For example, the oligonucleotide purity obtained from this oligonucleotide synthesis process using the 90 wt. % acetonitrile/10 wt. % toluene blend may have purity in the 92% to 100% purity range, and more particularly, in the 92.6% to 99.8% purity range. Therefore, it may be advantageous to utilize a blend of 90 wt. % acetonitrile/10 wt. % toluene as a wash solvent in the oligonucleotide synthesis process, as compared to pure acetonitrile.

In a second example, with this same second support resin and second test sequence (Test 18), a substantially pure mixture of 85 weight % acetonitrile and 15 weight % toluene is used as a wash solvent in the oligonucleotide synthesis process. In this case, it has been surprisingly found that the resulting synthesized oligonucleotide is slightly more pure than a synthesized oligonucleotide produced using pure acetonitrile as a wash solvent. For example, the oligonucleotide purity obtained from an oligonucleotide synthesis process using the 85 wt. % acetonitrile/15 wt. % toluene blend may have purity in the 92% to 96% purity range, and more particularly, in the 92.6% to 95.7% purity range. Therefore, it may be advantageous to utilize a blend of 85 wt. % acetonitrile/15 wt. % toluene as a wash solvent in the oligonucleotide synthesis process, as compared to pure acetonitrile.

In another example, with this same second support resin) and second oligonucleotide test sequence (Test 18), a substantially pure mixture of 80 weight % acetonitrile and 20 weight % toluene is used as a wash solvent in the oligonucleotide synthesis process. In this case, it has been surprisingly found that the resulting synthesized oligonucleotide is slightly more pure than a synthesized oligonucleotide produced using pure acetonitrile as a wash solvent. For example, the oligonucleotide purity obtained from an oligonucleotide synthesis process using the 80 wt. % acetonitrile/20 wt. % toluene blend may have purity in the 93% to 97% purity range, and more particularly, in the 93.9% to 96.9% purity range. Therefore, it may be advantageous to utilize a blend of 80 wt. % acetonitrile/20 wt. % toluene as a wash solvent in the oligonucleotide synthesis process, as compared to pure acetonitrile.

In yet another example, where a substantially pure mixture of the azeotropic composition of 76 weight % acetonitrile and 24 weight % toluene is used as a wash solvent in the oligonucleotide synthesis process using the second support resin and second oligonucleotide test sequence (T-18), it has been surprisingly found that the resulting synthesized oligonucleotide has higher purity than a synthesized oligonucleotide produced using pure acetonitrile as a wash solvent. For example, the purity of the oligonucleotide obtained from an oligonucleotide synthesis process using the 76 wt. % acetonitrile/24 wt. % toluene blend with the second resin and T18 sequence, may be in the 95%-97% range, and more particularly, with the purity in the 95.3% to 96.8% range. Therefore, it may be advantageous to utilize a blend of 76 wt % acetonitrile/24 wt % toluene as a wash solvent in the oligonucleotide synthesis process, as compared to pure acetonitrile.

Therefore, and as demonstrated in the foregoing exemplifications, it has been found that a blended wash solvent composition of 5-24 weight percent toluene with corresponding 95-76 weight percent acetonitrile results in at least the same purity levels of the resulting oligonucleotides produced by the oligonucleotide synthesis processes, if not a higher purity of such oligonucleotides, than pure acetonitrile wash solvent. Said another way, the foregoing exemplifications demonstrates that the use of a blended wash solvent comprising as little as 5 weight % toluene and as much as 24 weight % toluene, and as little as 76 weight % acetonitrile and as much as 95 weight % acetonitrile (or any range defined by any two of the foregoing values), in an oligonucleotide synthesis process results in a oligonucleotide purity at least as pure as a pure acetonitrile wash solvent, if not more pure.

As described previously, the generated purified acetonitrile and toluene mixture 126 may be purified to a substantially pure (e.g., 80% or more, and more preferably, 95% or more pure) mixture of acetonitrile and toluene, which may be at the azeotropic composition of acetonitrile and toluene or at an acetonitrile to toluene ratio that contains more acetonitrile than the azeotrope. For example, the generated purified acetonitrile and toluene mixture 126 may be purified to a substantially pure azeotropic composition of approximately 76 wt. % acetonitrile and 24 wt. % toluene. In this case, although it has been found that the reuse of this blended composition as a wash solvent in the oligonucleotide syntheses process may produce oligonucleotide purity at least similar to pure acetonitrile (e.g., reuse of the azeotropic composition is at least acceptable, if not preferable as compared to pure acetonitrile), it may be advantageous to increase the relative weight percent of the acetonitrile in the mixture in order to target the more advantageous compositions of the wash solvent, as described above. For example, it may be possible to increase the weight percent of the acetonitrile from the azeotropic composition of 76 weight percent, to 80 weight percent, and more preferably 85 weight percent, and possibly 90 weight percent, and in some cases to 95 weight percent, in order to meet the more advantageous acetonitrile and toluene blends as described previously.

Increasing the overall weight percent of the acetonitrile can be accomplish through adding acetonitrile into the generated purified acetonitrile and toluene mixture 126 at some point in the process prior to using the recycled purified acetonitrile and toluene mixture 126 in the oligonucleotide synthesis process as a wash solvent. For example, supplemental acetonitrile 154 may be added to purified acetonitrile and toluene mixture 126 (e.g., either in-line, or by any other suitable mixing device) prior to using the purified acetonitrile and toluene mixture 126 as a wash solvent blend in the oligonucleotide synthesis process. In this case, the amount of supplemental acetonitrile 154 added to purified acetonitrile and toluene mixture 126 may be based upon the amount of supplemental acetonitrile 154 needed to achieve a targeted composition of the blend of the acetonitrile and toluene. For example, the amount of supplemental acetonitrile 154 added to purified acetonitrile and toluene mixture 126 may be the amount needed to increase the weight percent of acetonitrile from the azeotropic composition (e.g., 76 wt. %) to 80 weight percent, and more preferably 85 weight percent, and possibly 90 weight percent, and in some cases to 95 weight percent acetonitrile. Alternatively, in the case where the relative weight percent of acetonitrile to toluene is greater than the azeotropic composition, the amount of supplemental acetonitrile 154 added to purified acetonitrile and toluene mixture 126 may be the amount needed to increase the relative weight percent of acetonitrile to 80 weight percent, and more preferably 85 weight percent, and possibly 90 weight percent, and in some cases to 95 weight percent acetonitrile. Additionally, although supplemental acetonitrile 154 is illustrated as being added to purified acetonitrile and toluene mixture 126, supplemental acetonitrile 154 could be added to any one of, or combination of, collection tank 104, feed stream 105, overhead fraction 108, purified acetonitrile and toluene stream 114, and/or purified acetonitrile and toluene stream 120, such that the composition of acetonitrile and toluene mixture 126 contains any of the above described weight percent's of acetonitrile. In any of these cases, the use of the resulting purified acetonitrile and toluene mixture 126 recycled into the oligonucleotide syntheses process as a wash solvent may increase the overall purity of the oligonucleotide syntheses process as compared to if pure acetonitrile was used as the wash solvent.

Although the foregoing discussion relates to the purification, possible acetonitrile supplementation, and recycling of a recovered waste stream from an oligonucleotide synthesis process for use as a wash solvent, it is to be understood that such discussion is not meant to limit the invention only to the context of waste stream recycling. As described previously, the use of the acetonitrile and toluene blend as a wash solvent has been found to be advantageous as compared to pure acetonitrile alone. Therefore, any of the processing steps relating to the purification and supplementation of waste stream 102 may not be necessary, and rather an oligonucleotide synthesis process utilizing a fresh (e.g., not recycled) stream of any of the acetonitrile and toluene compositions as described above, may be equally as advantageous, if not more advantageous, for use as a wash solvent in the oligonucleotide synthesis process, and is therefore also encompassed by this disclosure.

### Materials and Methods

### Examples 1-8

As relating to Examples 1-8 described below, azeotropic or close-boiling mixtures of acetonitrile and toluene waste containing iodine, thiols, alkylamines, and other impurities is being treated by addition of a reductant, such as sodium thiosulfate, silver nitrate, and/or formic acid, into the feed stream. In the case of sodium thiosulfate, the reductants convert all dissolved organic and inorganic iodine compounds to iodide, which is not volatile, and can be removed via side offtake without any need for additional equipment.

Unless indicated otherwise, all concentrations are by mass, i.e., percentages are weight percentages and ppm is ppm by weight. In all examples, the feed mass in the rectification column was 1 kg of a binary mixture consisting of 76% of acetonitrile and 24% of toluene.

Fractional distillation was performed in a 400 mm packed glass column equipped with a liquid divider and topped with a cooled outlet. The column itself is comprised of an evacuated (10-6 mbar) silver coated insulating jacket, sight strips and outer bellows (one bellow packing per 500 mm length). The packing consists of corrosion resistant Raschig rings made of borosilicate glass. The 40 cm packed column was operated at a reflux ratio of RR=0.33 and a gradually reducing pressure. The experiments were carried out so that 80% of the initial feed was obtained as overhead product. The distillate was divided in three cuts with 200 ml each.

As used herein, “artificial waste” refers to the composition shown below in Table 1.

TABLE 1

| Component | Amount (wt. %) |
|---|---|
| Acetonitrile | 80.0 |
| Toluene | 5.0 |
| Pyridine | 2.0 |
| 2,6-Lutidine | 1.0 |
| Picoline | 1.0 |
| N-Methyl imidazole | 1.0 |
| Acetic acid | 1.0 |
| Water | 1.0 |
| Phenylacetyl disulfide | 0.0020 |
| Dichloroacetic acid | 0.0010 |
| 5-Ethylthio-1H-tetrazole | 0.0010 |
| Iodine | 0.00005 |

The artificial waste may further include trace metals in an amount of less than 0.2 ppm. The trace metals may comprise one or more of aluminum, barium, bismuth, cadmium, calcium, chromium, cobalt, copper, iron, lead, lithium, magnesium, manganese, molybdenum, nickel, potassium, silver, sodium, strontium, tin, and zinc.

### Example 9: Test-20 Oligonucleotide (DNA and RNA)

Oligonucleotide (DNA and RNA) synthesis is generally conducted via a four step cycle (deblocking, activation/coupling, capping and oxidation/sulfurization) which is repeated for each nucleotide added until the desired sequence is obtained. Between each step, oligonucleotides bound to a support are washed with an acetonitrile-based wash solvent to reduce residual reagents from the prior step. The wash solvent is usually pure acetonitrile.

It was hypothesized that a blend of acetonitrile and toluene could be used as a wash solvent rather than pure acetonitrile alone. To test such hypothesis, various compositions of a substantially pure mixture of acetonitrile and toluene were utilized as wash solvents in multiple oligonucleotide syntheses reactions, which yielded various purities of the resulting oligonucleotide.

In this case, the following oligonucleotide syntheses test conditions were observed for the Test 20 (e.g., “20-mer”) Oligonucleotide:

| | |
|---|---|
| Oligonucleotide | AKTA Oligopilot 100 instrument (currently sold by |
| Synthesizer | Cytiva) |
| Primer Support | Polystyrene with Unylinker TM loading of 334 |
| (first resin) | micromole/gram |
| Synthesis Scale | 200 micromoles, 6.3 mL column |
| Oligonucleotide | Test-20 “20-mer,” synthetic oligonucleotide with |
| | 5′- and 3′-hydroxyl ends |
| DNA Amidites | dT, dA, dC, dG at 0.15M in acetonitrile, 1.8 |
| | equivalences used |
| Coupling Time | 3 minutes |
| Activator Reagent | 0.3M 5-benzythio-1H-tetrazole in acetonitrile |
| Deblock Reagent | 3% dichloroacetic acid in toluene (v/v) |
| Capping Reagents | 20% acetic anhydride, 30% 2,6-lutidine, 50% |
| | acetonitrile (v/v) |
| | 20% N-methylimidazole, 80% acetonitrile (v/v) |
| Oxidation | 0.05M Iodine, 10% water, 90% pyridine (v/v) |
| Reagent | |

The oligonucleotide yield was determined by HPLC using UV detection at 260 nm. In addition, the identity of the main peak was confirmed by LCMS.

### Example 10: Test-18 Oligonucleotide (DNA and RNA)

Similar to Example 9, Oligonucleotide (DNA and RNA) synthesis is generally conducted via a four step cycle (deblocking, activation/coupling, capping and oxidation/sulfurization) which is repeated for each nucleotide added until the desired sequence is obtained. Between each step, oligonucleotides bound to a support are washed with an acetonitrile-based wash solvent to reduce residual reagents from the prior step. The wash solvent is usually pure acetonitrile.

As with Example 9, it was hypothesized that a blend of acetonitrile and toluene could be used as a wash solvent rather than pure acetonitrile alone. To test such hypothesis, various compositions of a substantially pure mixture of acetonitrile and toluene were utilized as wash solvents in multiple oligonucleotide syntheses reactions, which yielded various purities of the resulting oligonucleotide.

In this case, the following oligonucleotide syntheses test conditions were observed for the Test 18 (e.g., “18-mer”) Oligonucleotide:

| | |
|---|---|
| Oligonucleotide | AKTA Oligopilot 100 instrument (currently sold |
| Synthesizer | by Cytiva) |
| Primer Support | Kinovate Polysterol dT (NPHL) 353 micromole/ |
| (second resin) | gram or Kinovate Polysterol dT (NPHL) 362 |
| | micromole/gram |
| Synthesis Scale | 200 micromoles, 6.3 mL column |
| Oligonucleotide | Test-18 “18-mer,” synthetic oligonucleotide with |
| | 5′- and 3′-hydroxyl ends |
| DNA Amidites | dT at 0.15M in acetonitrile, 1.8 equivalences used |
| Coupling Time | 3 minutes |
| Activator Reagent | 0.3M 5-benzythio-1H-tetrazole in acetonitrile |
| Deblock Reagent | 3% dichloroacetic acid in toluene (v/v) |
| Capping Reagents | 20% acetic anhydride, 30% 2,6-lutidine, 50% |
| | acetonitrile (v/v) |
| | 20% N-methylimidazole, 80% acetonitrile (v/v) |
| Oxidation | 0.05M Iodine, 10% water, 90% pyridine (v/v) |
| Reagent | |

The oligonucleotide yield was determined by HPLC using UV detection at 260 nm. In addition, the identity of the main peak was confirmed by LCMS.

### EXAMPLES

### Example 1: Fractional Distillation of Artificial Waste

In this example, 1000 ml of artificial waste further containing acetonitrile, toluene, tetrahydrofuran (THF), and organic agents from the oligonucleotide synthesis as described in table 01 was introduced into a round-bottom flask of the fractional distillation column. The fractional distillation was carried out without any pre-treatment additives or entrainer. Top and bottom temperatures were established at 76° C. and 85° C., respectively. The distillate was again condensed at the column head in a cooler and collected into two fractions. The first fraction (Cut 1) was of minimal volume. It contained 1-2% water as well as the components shown below in Table 2. The main fraction (Cut 2) was also collected and analyzed.

The distillate of the artificially compounded rinsing solution may be purified by a simple fractional distillation; however, various components of the chemicals used for oligonucleotide synthesis may carry over. Thus, in addition to water, significant proportions of the alkylamines, acetic acid, dichloroacetic acid, alkylthiols, and iodine used were found in both distillates. The results of the analysis of the artificial waste distillates are shown in Table 2, along with their specification values (“Spec.”).

TABLE 2

| Run 1 | Spec. | | Artificial Waste | Cut 1 | | Cut 2 | |
|---|---|---|---|---|---|---|---|
| Acetonitrile (100%) | Min. | 94% | 82.0% | 75.4% | | 95.9% | |
| Toluene | Max. | 5% | 5.0% | 9.2% | | 3.3% | |
| THF | Max. | 5% | 5.0% | 15.3% | | 0.03% | |
| Pyridine | <100 | ppm | 20.000 ppm | 80 | ppm | 6500 | ppm |
| 2,6-Lutidine | <100 | ppm | 10.000 ppm | 60 | ppm | 30 | ppm |
| Picoline | <100 | ppm | 10.000 ppm | 100 | ppm | 800 | ppm |
| N-Methyl imidazole | <100 | ppm | 10.000 ppm | <50 | ppm | <50 | ppm |
| Water | <1 | ppm | 10.000 | 8260 | ppm | 930 | ppm |
| Phenylacetyl disulfide | <5 | ppm | 200 | <0.5 | ppm | <0.5 | ppm |
| Dichloroacetic acid | <1 | ppm | 100 | <0.5 | ppm | <0.5 | ppm |
| 5-Ethylthio-1H-tetrazole | <1 | ppm | 100 | <0.5 | ppm | <0.5 | ppm |
| Iodine | <1 | ppm | 5 | <0.5 | ppm | <0.5 | ppm |

### Example 2: Fractional Distillation of Acetonitrile and Toluene with Added Iodine

In this Example, 200 mg of solid iodine was added to an azeotrope of 361 g acetonitrile and 114 g toluene (designated as “Azeotrope” in the table below). The mixture was stirred until the solid iodine was dissolved completely, then transferred to the round-bottom flask of a fractional distillation column. Top and bottom temperatures were established at 76° C. and 80° C., respectively. The distillate was condensed in a cooler and collected at the column head into equal fractions. It was noted that the condensate (Cuts 1-3) was still colored, and the smell of iodine could be perceived.

The cuts were analyzed, and the results are shown below in Table 3, along with the specification values (“Spec.”).

TABLE 3

| Run 2 | Spec. | | Azeotrope | Cut 1 | Cut 2 | Cut 3 |
|---|---|---|---|---|---|---|
| Acetonitrile | Min. | 94% | 76.0% | 75.40% | 95.90% | 97.3% |
| (100%) | | | | | | |
| Toluene | Max. | 5% | 24.0% | 9.20% | 3.30% | 3.30% |
| Iodine | <1 | ppm | 205 ppm | 2 ppm | 4 ppm | 8 ppm |

### Example 3: Precipitation of Iodine from Acetonitrile and Toluene Using Sodium Thiosulfate

In this Example, 200 mg of solid iodine and an equimolar amount (259 mg) of solid sodium thiosulfate were added to an azeotropic mixture of 361 g acetonitrile and 114 g toluene. The solution was stirred with a magnetic stirrer at 500 rpm until the solids were completely dissolved. Then, the distillation was performed, with the top and bottom temperatures established at 81° C. and 85° C., respectively. The distillate was condensed at the column head in a cooler and collected into two equal fractions.

In this advantageous version, the condensed distillate was colorless. The iodine content was below 0.1 ppm. The results of analysis of the cuts are shown below in Table 4, along with the specification values (“Spec.”).

TABLE 4

| Run 3 | Spec. | | Azeotrope | Cut 1 | Cut 2 |
|---|---|---|---|---|---|
| Acetonitrile (100%) | Min. | 94% | 76.0% | 75.40% | 95.90% |
| Toluene | Max. | 5% | 24.0% | 9.20% | 3.30% |
| Iodine | <1 | ppm | 205 ppm | <0.1 ppm | <0.1 ppm |

### Example 4: Precipitation of Iodine from Artificial Waste Using Sodium Thiosulfate

In this Example, 500 ml of artificial waste was spiked with 125 mg iodine and 160 mg sodium thiosulfate. The mixture, which further contained 65% acetonitrile and 25% toluene, was introduced into a round-bottom flask of the fractional distillation column. Top and bottom temperatures were established at 76° C. and 80° C., respectively. The distillate was again condensed at the column head in a cooler and collected into two fractions. The two main fractions were analyzed.

In contrast to the Example 3, 1.2 ppm iodide were found upon analysis of Cut 2. This indicates that the higher concentrations of iodine in the oligonucleotide waste requires an excess of reductant to prevent iodine carryover. The results of the analysis are shown below in Table 5, along with the specification values (“Spec.”).

TABLE 5

| | | | Artificial | | |
|---|---|---|---|---|---|
| Run 4 | Spec. | | Waste | Cut 1 | Cut 2 |
| Additional iodine | | | 250 ppm | | |
| Sodium thiosulfate | | | 320 ppm | | |
| Acetonitrile | Min. | 94% | 65.0% | 94.0% | 77.5% |
| Toluene | Max. | 5% | 25.0% | 5.0% | 22.2% |
| Pyridine | <100 | ppm | 24500 | 50 | 2600 |
| 2,6-Lutidine | <100 | ppm | 12200 | 100 | 80 |
| Picoline | <100 | ppm | 12200 | 100 | 220 |
| N-Methyl imidazole | <100 | ppm | 12200 | 100 | 50 |
| Acetic acid | <1 | ppm | 12200 | — | 8 |
| Water | <30 | ppm | 10000 | — | 1793 |
| Phenylacetyl disulfide | <5 | ppm | 20 | — | <0.5 |
| Dichloroacetic acid | <1 | ppm | 10 | — | <0.5 |
| 5-Ethylthio-1H-tetrazole | <1 | ppm | 10 | — | <0.5 |
| Iodine | <1 | ppm | 5 | — | 1.2 |

### Example 5: Precipitation of Thiols from Acetonitrile and Toluene Using Silver Nitrate

In this Example, 100 mg phenylacetyl disulfide (PAD) and 50 mg 5-ethylthio-1H-tetrazole (ETT) were added to an azeotrope of 380 g acetonitrile and 120 g toluene (designated as “Azeotrope” in the table below). Finally, to this mixture 51 mg silver nitrate was added. Then the distillation was performed, with the top and bottom temperatures established at 76° C. and 81° C., respectively. The distillate was condensed at the column head in a cooler and collected into two equal fractions.

In this advantageous version, the condensed distillate was colorless and without the intensive smell of sulfur compounds. The alkylthiol content was reduced to below the detection limit of the HPLC. The results of the analyses are shown below in Table 6 along with the specification values (“Spec.”).

TABLE 6

| | Analysis | | | | | |
|---|---|---|---|---|---|---|
| Run 5 | Method | Spec. | Azeotrope | Cut 1 | Cut 2 | Cut 3 |
| Acetonitrile | GC | | 76.0% | 87.2 | 77.6 | 74.3 |
| Toluene | GC | | 24.0% | 21.7 | 22.4 | 25.6 |
| Phenylacetyl | HPLC | <5 ppm | 0.020% | <0.5 | <0.5 | <0.5 |
| disulfide | | | | | | |
| 5-Ethylthio-1H- | HPLC | <1 ppm | 0.010% | <0.5 | <0.5 | <0.5 |
| tetrazole | | | | | | |

### Example 6: Precipitation of Alkylamines from Acetonitrile and Toluene Using Formic Acid

In this Example, the effectiveness of formic acid as a reducing agent and Lewis acid was investigated in order to precipitate alkylamines by forming Lewis adducts. To an azeotrope of 361 g acetonitrile and 114 g toluene were added 10 g pyridine, and 5 g each of picoline, 2,6-lutidine and N-methyl imidazole (designated as “Azeotrope” in the table below). Finally, to this mixture was added 13.24 g formic acid. Then, the distillation was performed, and the top and bottom temperatures were established at 81° C. and 85° C., respectively. The distillate was condensed at the column head in a cooler and collected into three equal fractions.

In this advantageous version, the condensed distillate was colorless. The carryover of the alkylamines had been significantly reduced, suggesting that the formic acid formed a non-volatile salt with the alkylamine bases, preventing them from vaporizing during distillation. The results of the analysis are shown below in Table 7, along with the specification values (“Spec.”).

TABLE 7

| | Analysis | | | | | | | | |
|---|---|---|---|---|---|---|---|---|---|
| Run 6 | Method | Spec. | | Azeotrope | Cut 1 | Cut 2 | Cut 3 | | Reduction % |
| Acetonitrile | GC | Min. | 94% | 76.0% | 78.4% | 78.1% | 76.5% | | |
| (100%) | | | | | | | | | |
| Toluene | GC | Max. | 5% | 24.0% | 21.3% | 21.8% | 23.3% | | |
| Pyridine | GC | <100 | ppm | 2.0% | 300 ppm | 500 pm | 1400 | ppm | 93.00% |
| 2,6-Lutidine | GC | <100 | ppm | 1.0% | <100 ppm | <100 | <101 | ppm | 100.00% |
| Picoline | GC | <100 | ppm | 1.0% | <100 ppm | <100 | 190 | ppm | 98.10% |
| N-Methyl | GC | <100 | ppm | 1.0% | <100 ppm | <100 | <101 | ppm | >99% |

Imidazole

### Example 7: Precipitation of Iodine from Artificial Waste Using Excess Sodium Thiosulfate

In this Example, 1000 ml artificial waste which further contained 5 ppm iodine and an equimolar amount of sodium thiosulfate was introduced into the round-bottom flask of the fractional distillation column. 12 mg sodium thiosulfate was added to reduce the organic iodine compounds in the waste solution. Top and bottom temperatures were established at 76° C. and 81° C., respectively. The distillate was again condensed at the column head in a cooler and collected into two fractions. The first fraction was of minimal volume. It contained 1-2% water as well as the components shown in Table 8. The main fraction was also collected and analyzed for iodine content. No iodine could be detected in the distillate within the detection limits of the method. The results of the analysis are shown below in Table 8, along with the specification values (“Spec.”).

TABLE 8

| Run 7 | Spec. | | Artificial Waste | | Cut 1 | | Cut 2 | |
|---|---|---|---|---|---|---|---|---|
| Sodium thiosulfate | | | 100 | ppm | | | | |
| Acetonitrile | Min. | 94% | 65.7% | | 94.0% | | 77.5% | |
| Toluene | Max. | 5% | 25.7% | | 5.0% | | 22.2% | |
| Pyridine | <100 | ppm | 24500 | ppm | 970 | ppm | 1700 | ppm |
| 2,6-Lutidine | <100 | ppm | 12200 | ppm | 110 | ppm | 200 | ppm |
| Picoline | <100 | ppm | 12200 | ppm | 250 | ppm | 500 | ppm |
| N-Methyl imidazole | <100 | ppm | 12200 | ppm | 100 | ppm | 50 | ppm |
| Acetic acid | <1 | ppm | 12200 | ppm | 12 | ppm | 25 | ppm |
| Water | <30 | ppm | 10000 | ppm | 930 | ppm | 1370 | ppm |
| Phenylacetyl disulfide | <5 | ppm | 20 | ppm | 12 | ppm | <0.5 | ppm |
| Dichloroacetic acid | <1 | ppm | 10 | ppm | <3 | ppm | <3 | ppm |
| 5-Ethylthio-1H-tetrazole | <1 | ppm | 10 | ppm | <0.5 | ppm | <0.5 | ppm |
| Iodine | <1 | ppm | 5 | ppm | <0.5 | ppm | <0.5 | ppm |

### Example 8: Precipitation of Iodine and Alkylamines from Artificial Waste

In this Example, 500 ml artificial waste, 10 mg sodium thiosulfate and an excess of formic acid were mixed. The mixture, which further contained 65% acetonitrile and 25% toluene, was introduced into the round-bottom flask of the fractional distillation column. Top and bottom temperatures were established at 76° C. and 81° C., respectively. The distillate was again condensed at the column head in a cooler and collected into two fractions.

The concentration of all alkylamines tested was below the specification limit, and the amount of iodine in the distillate was below 0.5 ppm (the detection limit of the HPLC method). The results of the analysis are shown below in Table 9, along with the specification values (“Spec.”).

TABLE 9

| Run 8 | Spec. | | Artificial Waste | | Cut 1 | | Cut 2 | |
|---|---|---|---|---|---|---|---|---|
| Acetonitrile | Min. | 94% | 82.0% | | 62.9% | | 83% | |
| Toluene | Max. | 5% | 5.0% | | 9% | | 11.6% | |
| THF | Max. | 5% | 5.0% | | 28.1% | | 5.4% | |
| Pyridine | <100 | ppm | 20.000 | ppm | <50 | ppm | <50 | ppm |
| 2,6-Lutidine | <100 | ppm | 10.000 | ppm | <50 | ppm | <50 | ppm |
| Picoline | <100 | ppm | 10.000 | ppm | <50 | ppm | <50 | ppm |
| N-Methyl imidazole | <100 | ppm | 10.000 | ppm | 100 | ppm | <50 | ppm |
| Phenylacetyl disulfide | <5 | ppm | 200 | ppm | <0.5 | ppm | <0.5 | ppm |
| Dichloroacetic acid | <1 | ppm | 100 | ppm | <0.5 | ppm | <0.5 | ppm |
| 5-Ethylthio-1H-tetrazole | <1 | ppm | 100 | ppm | <0.5 | ppm | <0.5 | ppm |
| Iodine | <1 | ppm | 5 | ppm | <0.5 | ppm | <0.5 | ppm |

### Example 9: Test-20 Oligonucleotide Purities Attained Though Use of Various Wash Solvent Compositions

In this example, four different compositions of acetonitrile-based wash solvents were used to synthesize the same Test-20 20-mer oligonucleotide, over four testing runs, under the experimental conditions provided previously. The first test utilized a pure (e.g., 100 wt. %) acetonitrile wash solvent, the second test utilized a wash solvent comprising 95 wt % acetonitrile and 5 wt. % toluene, the third test utilized a wash solvent comprising 85 wt % acetonitrile and 15 wt. % toluene, and the fourth test utilized a wash solvent comprising 76 wt % acetonitrile and 24 wt. % toluene (e.g., representative of an azeotropic composition of acetonitrile and toluene). The purity of the resulting 20-mer oligonucleotide was reported based upon HPLC yield, and the results represented in Table 10 below:

TABLE 10

| | | | Standard |
|---|---|---|---|
| Wash Solvent wt. | Wash Solvent wt. | Average | Deviation of |
| % Acetonitrile | % Toluene | HPLC Yield | HPLC Yield |
| 100 | 0 | 70.97 | 0.88 |
| 95 | 5 | 72.15 | 1.26 |
| 85 | 15 | 74.75 | 1.65 |
| 76 | 24 | 71.75 | 0.36 |

It has been surprisingly found that there is a slightly increased yield of the Test-20 oligonucleotide product formed during the solid phase chemical synthesis of oligonucleotides when utilizing a less-than-pure acetonitrile wash solvent. Specifically, it is observed that the use of a blended composition containing 85% acetonitrile and 15% toluene (w/w) wash solvent resulted in the highest oligonucleotide yield. However, in each of the foregoing cases, the blended compositions of the acetonitrile and toluene wash solvents exhibited at least similar, if not higher test-20 oligonucleotide yields than that of a pure acetonitrile wash solvent. As such, this experiment confirmed that the use of blended wash solvents rather than pure acetonitrile, in an oligonucleotides synthesis process is not only possible (e.g., oligonucleotide synthesis not necessarily requiring pure acetonitrile wash solvents), but may be advantageous.

### Example 10: Test-18 Oligonucleotide Purities Attained Though Use of Various Wash Solvent Compositions

In another example, five different compositions of acetonitrile-based wash solvents were used to synthesize the Test-18 18-mer oligonucleotide, over five testing runs, under the experimental conditions provided previously. The first test utilized a pure (e.g., 100 wt. %) acetonitrile wash solvent, the second test utilized a wash solvent comprising 90 wt % acetonitrile and 10 wt. % toluene, the third test utilized a wash solvent comprising 85 wt % acetonitrile and 15 wt. % toluene, the fourth test utilized a wash solvent comprising 80 wt % acetonitrile and 20 wt. % toluene and the fifth test utilized a wash solvent comprising 76 wt % acetonitrile and 24 wt. % toluene (e.g., representative of an azeotropic composition of acetonitrile and toluene). The purity of the resulting 18-mer oligonucleotide was reported based upon HPLC yield, and the results represented in Table 11 below:

TABLE 11

| | | | Standard |
|---|---|---|---|
| Wash Solvent wt. | Wash Solvent wt. | Average | Deviation of |
| % Acetonitrile | % Toluene | HPLC Yield | HPLC Yield |
| 100 | 0 | 92.19 | 0.95 |
| 90 | 10 | 95.37 | 3.89 |
| 85 | 15 | 93.98 | 1.55 |
| 80 | 20 | 95.25 | 1.49 |
| 76 | 24 | 93.18 | 2.07 |

It has been surprisingly found that there is a slightly increased yield of the T-18 oligonucleotide product formed during the solid phase chemical synthesis of oligonucleotides when utilizing a less-than-pure acetonitrile wash solvent. Specifically, it is observed that the use of a blended composition containing 80% acetonitrile and 20% toluene (w/w) wash solvent resulted in the highest oligonucleotide yield. However, in each of the foregoing cases, the blended compositions of the acetonitrile and toluene wash solvents exhibited at least similar, if not higher T-18 oligonucleotide yields than that of a pure acetonitrile wash solvent. As such, this experiment confirmed that the use of blended wash solvents rather than pure acetonitrile, in an oligonucleotide synthesis process is not only possible (e.g., oligonucleotide synthesis not necessarily requiring pure acetonitrile wash solvents), but may be advantageous.

### Aspects

Aspect 1 is a method for processing a waste stream, the method comprising: receiving the waste stream comprising acetonitrile, toluene, and one or more iodine containing compounds; adding an iodine reactive compound to the waste stream; fractionating the waste stream to produce an overhead fraction and a bottom fraction, the overhead fraction comprising the acetonitrile and the toluene of the waste stream, and the bottom fraction comprising the one or more iodine containing compounds of the waste stream.

Aspect 2 is a method of Aspect 1, wherein the iodine reactive compound comprises sodium thiosulfate.

Aspect 3 is a method of any of Aspects 1 or 2, wherein the iodine reactive compound comprises sodium thiosulfate and silver nitrate.

Aspect 4 is a method of any of Aspects 1 through 3, wherein the top fraction further comprises iodine at a concentration less than 25 ppm.

Aspect 5 is a method of any of Aspects 1 through 4, wherein the top fraction further comprises iodine at a concentration less than 1 ppm.

Aspect 6 is a method of any of Aspects 1 through 5, wherein, during the fractionation of the waste stream, the iodine reactive compound reacts with iodine of the one or more iodine containing compounds, and precipitates the iodine of the one or more iodine containing compounds from the waste stream into the bottom fraction.

Aspect 7 is a method of any of Aspects 1 through 6, further comprising purifying the overhead fraction to a purified overhead fraction consisting essentially of acetonitrile and toluene.

Aspect 8 is a method of any of Aspects 1 through 7, wherein the waste stream further comprises one or more sulfur containing compounds, the method further comprises adding a sulfur reactive compound to the waste stream, and the bottom fraction further comprises the one or more sulfur containing compounds.

Aspect 9 is a method of any of Aspects 1 through 8, wherein the sulfur reactive compound comprises silver nitrate.

Aspect 10 is a method of any of Aspects 1 through 9, wherein the waste stream further comprises one or more basic nitrogen containing compounds, the method further comprises adding an acidic reactive compound to the waste stream, and the bottom fraction further comprises the one or more basic nitrogen containing compounds.

Aspect 11 is a method of any of Aspects 1 through 10, wherein the acidic reactive compound comprises formic acid.

Aspect 12 is a method for processing a waste stream, the method comprising: receiving the waste stream comprising acetonitrile, toluene, and one or more sulfur containing compounds; adding a sulfur reactive compound to the waste stream; fractionating the waste stream to produce an overhead fraction and a bottom fraction, the overhead fraction comprising the acetonitrile and toluene of the waste stream, and the bottom fraction comprising the one or more sulfur containing compounds of the waste stream

Aspect 13 is a method of any of Aspects 1 through 11 or Aspect 12, wherein the top fraction further comprises sulfur at a concentration less than twenty five ppm.

Aspect 14 is a method of any of Aspects 1 through 11 or Aspect 12 through 13, wherein the top fraction further comprises sulfur at a concentration less than one ppm.

Aspect 15 is a method of any of Aspects 1 through 11 or Aspect 12 through 14, wherein the sulfur reactive compound comprises silver nitrate.

Aspect 16 is a method of any of Aspects 1 through 11 or Aspect 12 through 15, wherein, during the fractionation of the waste stream, the sulfur reactive compound reacts with the sulfur of the one or more sulfur containing compounds and precipitates the sulfur from the waste stream into the bottom fraction.

Aspect 17 is a method for processing a waste stream, the method comprising: receiving the waste stream comprising acetonitrile, toluene, and basic nitrogen containing compounds; adding an acidic reactive compound to the waste stream; fractionating the waste stream to produce an overhead fraction and a bottom fraction, the overhead fraction comprising the acetonitrile and toluene of the waste stream and the bottom fraction comprising the basic nitrogen containing compounds of the waste stream.

Aspect 18 is a method of any of Aspects 1 through 11, Aspects 12 through 16, or Aspect 17, wherein the top fraction further comprises one or more nitrogen containing compounds at a concentration less than one hundred ppm.

Aspect 19 is a method of any of Aspects 1 through 11, Aspects 12 through 16, or Aspects 17 and 18, wherein the acidic reactive compound comprises formic acid.

Aspect 20 is a method of any of Aspects 1 through 11, Aspects 12 through 16, or Aspects 17 through 19, wherein, during the fractionation of the waste stream, the acidic reactive compound reacts with the basic nitrogen containing compounds and precipitates the basic nitrogen containing compounds from the waste stream into the bottom fraction.

Aspect 21 is a method for producing a synthetic oligonucleotide comprising: washing a reaction vessel containing one or more components of an oligonucleotide synthesis process with a blended wash solution including at least acetonitrile and toluene, the acetonitrile comprising at least 70% of the blended wash solution; and recovering the synthetic oligonucleotide, the synthetic oligonucleotide comprising a minimum oligonucleotide purity.

Aspect 22 is a method of Aspect 21, wherein the blended wash solution comprises a substantially pure mixture of acetonitrile and toluene.

Aspect 23 is a method of any of Aspects 21 and 22, wherein the blended wash solution is received as a purified waste stream recycled from the oligonucleotide synthesis process.

Aspect 24 is a method of any of Aspects 21 through 23, wherein the substantially pure mixture of acetonitrile and toluene comprises at least 80% acetonitrile

Aspect 25 is a method of any of Aspects 21 through 24, wherein the substantially pure mixture of acetonitrile and toluene comprises at least 90% acetonitrile.

Aspect 26 is a method of any of Aspects 21 through 24, wherein the substantially pure mixture of acetonitrile and toluene comprises an azeotropic mixture of acetonitrile and toluene.

Aspect 27 is a method of any of Aspects 21 through 26, wherein the substantially pure mixture of acetonitrile and toluene comprises at least 76% acetonitrile.

Aspect 28 is a method of any of Aspects 21 through 27, wherein the substantially pure mixture of acetonitrile and toluene comprises at least 5% toluene.

Aspect 29 is a method of any of Aspects 21 through 28, wherein the substantially pure mixture of acetonitrile and toluene comprises at least 15% toluene.

Aspect 30 is a method of any of Aspects 21 through 29, wherein the minimum oligonucleotide purity is no less than 70% purity.

Aspect 31 is a method of any of Aspects 21 through 30, wherein the minimum oligonucleotide purity is greater than 72% purity.

Aspect 32 is a method of any of Aspects 21 through 31, wherein the minimum oligonucleotide purity is greater than 75% purity.

Aspect 33 is a method of any of Aspects 21 through 32, wherein the minimum oligonucleotide purity is no less than 90% purity.

Aspect 34 is a method of any of Aspects 21 through 33, wherein the minimum oligonucleotide purity is greater than 92% purity.

Aspect 35 is a method of any of Aspects 21 through 34, wherein the minimum oligonucleotide purity is greater than 95% purity.

Aspect 36 is a method of any of Aspects 21 through 35, wherein the minimum oligonucleotide purity is greater than an oligonucleotide purity produced by washing the reaction vessel containing the one or more components of an oligonucleotide synthesis process with a wash solution consisting of acetonitrile.

Aspect 37 is a method of any of Aspects 21 through 36 which incorporates any of the features of any of Aspects 1 through 11, Aspects 12 through 16, or Aspects 17 through 20.

Aspect 38 is a method of any of Aspects 1 through 11 that includes any of the features of any of Aspects 21 through 36, Aspects 12 through 16, or Aspects 17 through 20.

Aspect 39 is method of any of Aspects 12 through 16 that includes any of the features of any of Aspects 21 through 36, Aspects 1 through 11, or Aspects 17 through 20.

Aspect 40 is a method of any of Aspects 17 through 20 that includes any of the features of any of Aspects 21 through 36, Aspects 1 through 11, or Aspects 12 through 16.

What is claimed is:

1. A method for processing a waste stream, the method comprising:
receiving the waste stream comprising acetonitrile, toluene, and one or more iodine containing compounds;
adding an iodine reactive compound to the waste stream;
fractionating the waste stream to produce an overhead fraction and a bottom fraction, the overhead fraction comprising the acetonitrile and the toluene of the waste stream, and the bottom fraction comprising the one or more iodine containing compounds of the waste stream.

2. The method of claim 1, wherein the iodine reactive compound comprises sodium thiosulfate.

3. The method of claim 1, wherein the iodine reactive compound comprises sodium thiosulfate and silver nitrate.

4. The method of claim 1, wherein the top fraction further comprises iodine at a concentration less than 25 ppm.

5. The method of claim 1, wherein, during the fractionation of the waste stream, the iodine reactive compound reacts with iodine of the one or more iodine containing compounds, and precipitates the iodine of the one or more iodine containing compounds from the waste stream into the bottom fraction.

6. The method of claim 1, further comprising purifying the overhead fraction to a purified overhead fraction consisting essentially of acetonitrile and toluene.

7. A method for processing a waste stream, the method comprising:
receiving the waste stream comprising acetonitrile, toluene, and one or more sulfur containing compounds;
adding a sulfur reactive compound to the waste stream; and
fractionating the waste stream to produce an overhead fraction and a bottom fraction, the overhead fraction comprising the acetonitrile and toluene of the waste stream, and the bottom fraction comprising the one or more sulfur containing compounds of the waste stream.

8. The method of claim 7, wherein the top fraction further comprises sulfur at a concentration less than twenty-five ppm.

9. The method of claim 7, wherein the sulfur reactive compound comprises silver nitrate.

10. A method for processing a waste stream, the method comprising:
receiving the waste stream comprising acetonitrile, toluene, and basic nitrogen containing compounds;
adding an acidic reactive compound to the waste stream;
fractionating the waste stream to produce an overhead fraction and a bottom fraction, the overhead fraction comprising the acetonitrile and toluene of the waste stream and the bottom fraction comprising the basic nitrogen containing compounds of the waste stream.

11. The method of claim 10, wherein the top fraction further comprises one or more nitrogen containing compounds at a concentration less than one hundred ppm.

12. The method of claim 10, wherein the acidic reactive compound comprises formic acid.

13. A method for producing a synthetic oligonucleotide comprising:
washing a reaction vessel containing one or more components of an oligonucleotide synthesis process with a blended wash solution including at least acetonitrile and toluene, the acetonitrile comprising at least 70% of the blended wash solution; and
recovering the synthetic oligonucleotide, the synthetic oligonucleotide comprising a minimum oligonucleotide purity.

14. The method of claim 13, wherein the blended wash solution comprises a substantially pure mixture of acetonitrile and toluene.

15. The method of claim 13, wherein the blended wash solution is received as a purified waste stream recycled from the oligonucleotide synthesis process.

16. The method of claim 15, wherein the substantially pure mixture of acetonitrile and toluene comprises at least 80% acetonitrile.

17. The method of claim 15, wherein the substantially pure mixture of acetonitrile and toluene comprises at least 90% acetonitrile.

18. The method of claim 15, wherein the substantially pure mixture of acetonitrile and toluene comprises an azeotropic mixture of acetonitrile and toluene.

19. The method of claim 15, wherein the substantially pure mixture of acetonitrile and toluene comprises at least 5% toluene.

20. The method of claim 15, wherein the substantially pure mixture of acetonitrile and toluene comprises at least 15% toluene.
